(19) United States
(12) Patent Application Publication
(10) Pub. No.: US 2025/0099181 A1
(43) Pub. Date: Mar. 27, 2025

(54) MEDICAL DEVICES AND RELATED METHODS

(71) Applicant: Boston Scientific Scimed, Inc., Maple Grove, MN (US)

(72) Inventors: Kian S. LIM, Shrewsbury, MA (US); John SUBASIC, Stow, MA (US); Longquan CHEN, Lexington, MA (US); Niraj Prasad RAUNIYAR, Plymouth, MN (US)

(73) Assignee: Boston Scientific Scimed, Inc., Maple Grove, MN (US)

(21) Appl. No.: 18/896,357

(22) Filed: Sep. 25, 2024

Related U.S. Application Data
(60) Provisional application No. 63/585,330, filed on Sep. 26, 2023.

(51) Int. Cl. A61B 18/26; A61B 18/00; A61B 18/20; A61B 18/22

(52) U.S. Cl. CPC A61B 18/26; A61B 2018/00982; A61B 2018/2065; A61B 2018/225

(57) ABSTRACT

Medical devices and related methods are described, including a medical device that includes a handle including a housing. The housing may include a grip and an actuator. The handle may further include an imaging module including a printed circuit board, and a laser module including a first laser source configured to couple to a first laser fiber and a second laser source configured to couple to a second laser fiber. The laser module and the imaging module may be contained within the housing or the imaging module, the laser module, or both the imaging module and the laser module may be selectively attachable to and detachable from the housing.

### CROSS-REFERENCE TO RELATED APPLICATIONS

This application claims the benefit of priority to U.S. Provisional Application No. 63/585,330, filed on Sep. 26, 2023, which is incorporated by reference herein in its entirety.

### TECHNICAL FIELD

Various aspects of this disclosure relate generally to medical devices and methods of use thereof. In particular, medical devices and methods useful for procedures that include imaging and use of laser fiber optics.

### BACKGROUND

Laser devices and systems are used during various medical procedures, such as ureteroscopic and/or endoscopic procedures. For example, laser fibers may be used for kidney stone size estimation and/or to break up kidney stones. Laser devices and systems are often expensive, and the distance traversed by optical fibers from a laser source to the area of treatment can result in distortion.

### SUMMARY

According to some aspects of the present disclosure, a medical device may include a handle including a housing that may include a grip and an actuator. The handle may further include an imaging module including a printed circuit board. The handle may include a laser module including a first laser source configured to couple to a first laser fiber and a second laser source configured to couple to a second laser fiber. The laser module and the imaging module may be contained within the housing or the imaging module, the laser module, or both the imaging module and the laser module may be selectively attachable to and detachable from the housing.

According to some aspects, the medical device may further include a shaft extending from the handle to a distal tip. The distal tip may include an imaging device and a light source each operably coupled to the imaging module. In some examples, the medical device may further include the first laser fiber and the second laser fiber. The first laser fiber may be coupled to the first laser source and the second laser fiber may be coupled to the second laser source, and each of the first laser fiber and the second laser fiber may extend distally through the shaft to the distal tip. In some examples, the handle may include a port and the shaft may include a lumen in fluid communication with the port and the lumen may terminate at a distalmost face of the distal tip. In some examples, the laser module and the imaging module may be contained within the housing, and the laser module may further include a first coupler at least partially surrounding the first laser source and/or a second coupler at least partially surrounding the second laser source. In some examples, the handle may include a user element selectable by a user to control a first wavelength of the first laser source and a second wavelength of the second laser source. In some examples, the user element may include a knob, switch, or button configured to select the first wavelength and the second wavelength, and the second wavelength may be different from the first wavelength. In some examples, the first wavelength may be red light, green light, or blue light. In some examples, the imaging module and the laser module may be coupled to, and detachable from, the housing. In some examples, the imaging module and the laser module may be contained within a single unit that is selectively attachable to and detachable from the housing. The single unit may be coupled to, and detachable from, a portion of the housing distal to the grip. The single unit may form an angle of 90 degrees or less relative to a longitudinal axis of the housing when the single unit is attached to the housing. The imaging module and the laser module may be contained within separate units, each unit being coupled to, and detachable from, the housing independent of the other unit.

According to some aspects, the medical device may include a handle including a housing including a grip, a port, and an actuator. The handle may include an imaging module coupled to, and selectively detachable from, the housing, and may include a laser module coupled to, and selectively detachable from, the housing, the laser module comprising a first laser source and a second laser source. The medical device may further include a shaft extending from the handle to a distal tip, the shaft may include a lumen in fluid communication with the port, and the actuator may be configured to control movement of the shaft. In some examples, the distal tip of the shaft may include an imaging device and a light source each operably coupled to the imaging module, and the lumen of the shaft may terminate at the distal tip. In some examples, the first laser source may be coupled to a first laser fiber and the second laser source may be coupled to a second laser fiber, and each of the first laser fiber and second laser fiber may extend through the shaft to respective openings of the distal tip. In some examples, the imaging module and the laser module may be contained within separate units, each unit being independently detachable from the housing. In some examples, the imaging module and the laser module may be contained within a single unit coupled to, and detachable from, the housing.

According to some aspects, the medical device may include a handle including a housing including a grip and an actuator. The handle may include an imaging module coupled to, and selectively detachable from, the housing, and may include a laser module coupled to, and selectively detachable from, the housing, the laser module comprising a first laser source and a second laser source. The medical device may further include a shaft extending from the handle to a distal tip that may include an imaging device and a light source. The imaging device and the light source may be operably coupled to the imaging module and the actuator may be configured to control movement of the shaft. In some examples, the first laser source may be coupled to a first laser fiber and the second laser source may be coupled to a second laser fiber, and each of the first laser fiber and second laser fiber may extend through the shaft to the distal tip.

### BRIEF DESCRIPTION OF FIGURES

The accompanying drawings, which are incorporated in and constitute a part of this application, illustrate exemplary aspects of this disclosure and together with the description, serve to explain the principles of the present disclosure.

FIGS. 1A-1F illustrate an exemplary medical device, in accordance with some aspects of this disclosure.

FIG. 2 illustrates another exemplary medical device, in accordance with some aspects of this disclosure.

FIGS. 3A and 3B illustrate a component useful for exemplary medical devices, in accordance with some aspects of this disclosure.

FIGS. 4A and 4B illustrate an exemplary medical device, in accordance with some aspects of this disclosure.

### DETAILED DESCRIPTION

Particular aspects of the present disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body.

As used herein, the terms “comprises,” “comprising,” “including,” “includes,” “having,” “has” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.”

Further, relative terms such as, for example, “about,” “substantially,” “approximately,” etc., are used to indicate a possible variation of +10% in a stated numeric value or range.

Although ureteroscopes and endoscopes are referenced herein for illustration purposes, it will be appreciated that the disclosure encompasses any suitable medical device configured to allow an operator to access and view internal body anatomy of a subject (e.g., patient) and/or to deliver medical instruments, such as, for example, biopsy forceps, graspers, baskets, snares, probes, scissors, retrieval devices, lasers, and other tools, into the subject's body. The medical devices herein may be inserted into a variety of body lumens and/or cavities, such as, for example, the urinary tract or gastrointestinal tract. It will be appreciated that, unless otherwise specified, bronchoscopes, duodenoscopes, endoscopes, gastroscopes, endoscopic ultrasonography (“EUS”) scopes, colonoscopes, ureteroscopes, bronchoscopes, laparoscopes, cystoscopes, aspiration scopes, sheaths, catheters, or any other suitable delivery device or medical device may be used in connection with the features described herein.

Features of the medical devices herein may help to reduce or minimize distortion of an output laser beam (or laser light) and/or reduce loss of output laser beam integrity. According to some aspects of the present disclosure, the medical device may include a laser module and an imaging module. The laser module may include one or more laser sources, e.g., housed within a handle of the medical device or detachably coupled to the handle. The laser module and the imaging module may be separate components, or may be incorporated together as a single component (e.g., each module including electronic components mounted to or otherwise coupled to a common substrate). The laser source(s) may be coupled to or configured to be coupled to laser fiber(s) extending through a shaft of the medical device to a distal end of the medical device, e.g., for treating a target site of a body lumen. The laser fibers may be, for example, Holmium fibers or other suitable optical fibers. The medical device may include one or more actuators to allow a user to control the laser source(s) and/or to select wavelength(s) of the laser source(s) during a medical procedure. The imaging module may include a printed circuit board (PCB). The PCB may include or be operably coupled to a processor, and may be configured to couple with a display external to the medical device for display of images. The PCB may electronically connect the processor and various imaging components using conductive tracks, solder pads, etc.

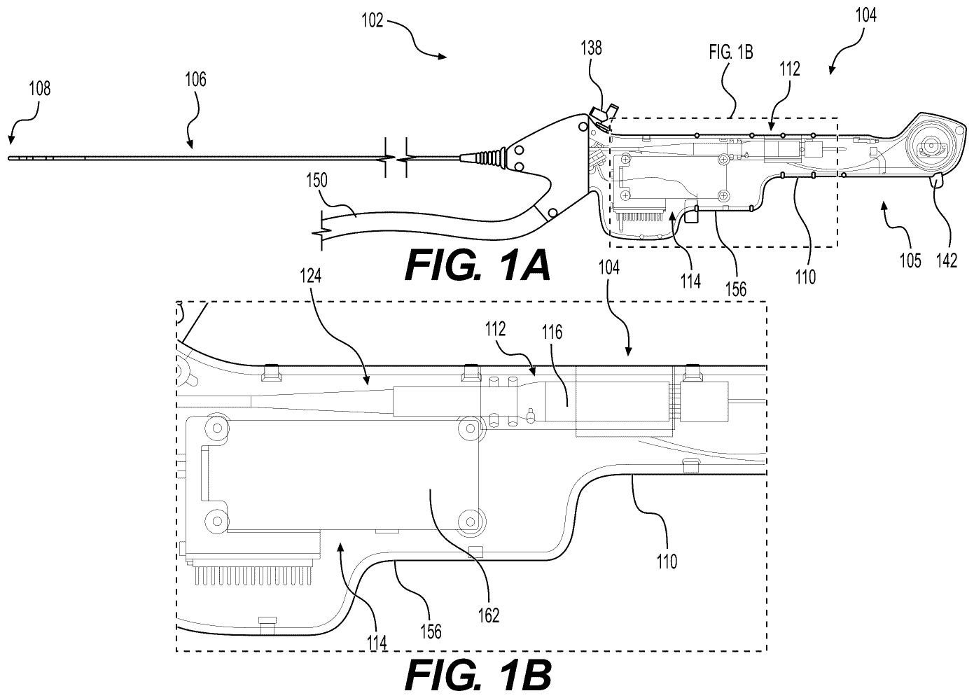

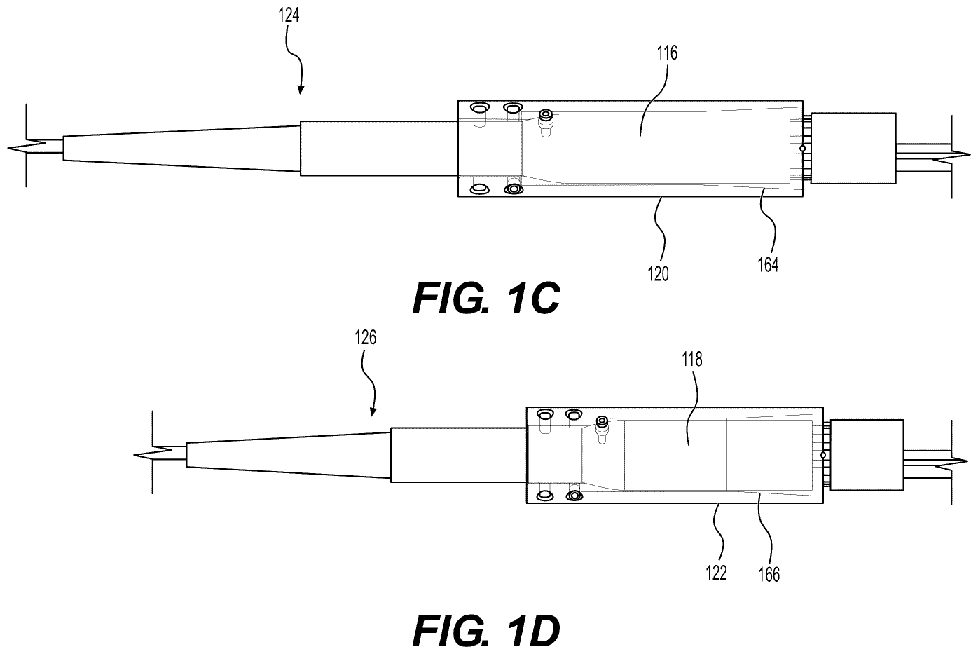

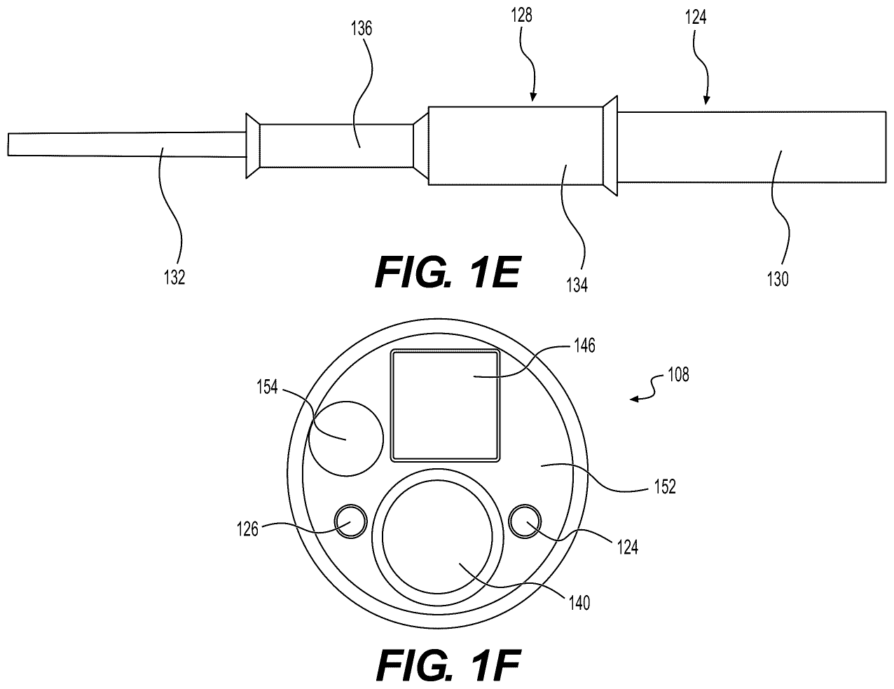

An exemplary medical device 102 illustrating some of the aspects of the present disclosure is shown in FIG. 1A. In this example, the device includes an imaging module and a laser module both contained within the housing of the handle (e.g., integrated into the handle, not detachable from the housing).

Medical device 102 includes a handle 104 and a shaft 106 coupled to handle 104. Handle 104 includes a housing 110 shown in FIGS. 1A and 1B, wherein the housing 110 contains various electronic components. In some examples, handle 104 is integral with shaft 106. In other examples, handle 104 is removably coupled to or otherwise detachable from shaft 106. Shaft 106 may be flexible to facilitate navigation through tortuous anatomical passages in the body. Shaft 106 may include one or more lumens and a distal end portion that terminates at a distal tip 108. Distal tip 108 may include imaging components such as an imaging device 146 (e.g., camera, imager, etc.), and one or more light sources 154 (e.g., LEDs, fiber optics, etc.) coupled to a PCB 162 of an imaging module 114. FIG. 1F shows a distalmost face 152 of distal tip 108 as including a distal opening of a lumen 140, imaging device 146, and light source 154. Handle 104 may include a port 138 in communication with lumen 140 of shaft 106, e.g., for insertion of an instrument and/or deliver suction or fluid via the distal opening at distalmost face 152.

Handle 104 may include one or more actuators for controlling aspects of medical device 102, e.g., via user input. The one or more actuators may include, for example, a lever, switch, button, knob, and/or any other suitable type of mechanism for receiving and transmitting user input. For example, housing 110 may include an actuator 142 at a proximal portion 105 of handle 104 to facilitate articulation, steering, deflection, and/or other movement of shaft 106 and distal tip 108. One or more other actuators of handle 104 may be configured to capture video and/or still images using imaging device 146 at distal tip 108 and/or to control laser sources 116, 118. A portion of housing 110 may provide a grip 156 to allow an operator to grasp the handle 104 during a medical procedure. Handle 104 may be operably coupled to one or more auxiliary devices, such as, e.g., a controller, a processor, a display, a power supply, etc., such as, via an umbilicus 150. Umbilicus 150 may be configured to deliver power to handle 104 and/or shaft 106 to power one or more components of handle 104 and/or shaft 106.

A laser module 112 may include a first laser source 116 (shown in FIGS. 1B and 1C) and a second laser source 118 (shown in FIG. 1D). Each laser source 116, 118 may be configured to emit one or more wavelengths of light, e.g., visible light. For example, each laser source 116, 118 may be capable of generating red, green, and/or blue light. Laser sources 116, 118 may be electrically coupled to a power source, e.g., via umbilicus 150.

Optionally, couplers 120, 122 (shown in side-view in FIGS. 1C and 1D) may partially or completely surround laser sources 116, 118. Couplers 120, 122 may each include a lumen to receive each laser source and/or couple each laser source to a respective laser fiber. As shown, for example, couplers 120, 122 define lumens 164, 166 each having an open proximal end to receive laser sources 116, 118 and an open distal end to receive a laser fiber 124, 126. Optionally, couplers 120, 122 may be secured to housing 110 by any suitable adhesive or fastener.

Laser fibers 124, 126 may extend from laser sources 116, 118 through shaft 106, e.g., to distalmost face 152 of distal tip 108. In some examples, each laser fiber 124, 126 may include a single, continuous, monolithic piece of fiber. In some examples, one or both laser fibers 124, 126 may include multiple fiber pieces in optical communication (e.g., joined by couplers, connectors, or fused together). For example, as shown in FIG. 1E, first laser fiber 124 may include a first fiber piece 130 and a second fiber piece 132 joined together with a fiber-to-fiber connector 128. Connector 128 may include a proximal section 134 having a larger outer diameter than distal section 136 of connector 128. Proximal section 134 may receive first fiber piece 130 and distal section 136 may receive second fiber piece 132 to optically couple fiber pieces 130, 132.

As shown in FIG. 1F, laser fibers 124, 126 may extend to corresponding holes or apertures on distalmost face 152 of distal tip 108. In this example, distal tip 108 is configured so that laser fibers 124, 126 are on diametrically opposite sides of lumen 140. However, it will be appreciated that other configurations are contemplated herein.

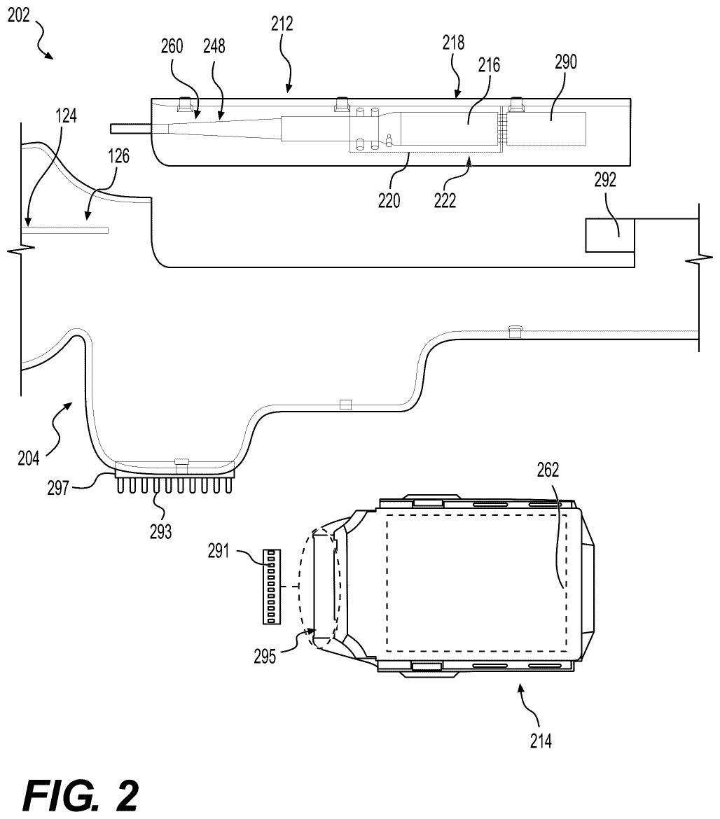

As mentioned above, the laser module and/or imaging module may be detachably coupled to the handle of a medical device. FIG. 2 illustrates an exemplary handle 204 of a medical device 202 according to aspects of the present disclosure, wherein a laser module 212 and an imaging module 214 are each detachable from handle 204. Handle 204 may be used in combination with a shaft, e.g., shaft 106 of medical device 102 (e.g., in place of handle 104), or may be used in combination with any of the features of shaft 106 of medical device 102. Furthermore, handle 204 may include any of the features of handle 104 (e.g., port 138, actuator 142, etc.), unless otherwise specified herein.

Similar to laser module 112, laser module 212 may include two laser sources 216, 218. FIG. 2 shows a side view of laser module 212 detached from handle 204, second laser source 218 being behind first laser source 216. The portion(s) of handle 204 to which laser module 212 attach may have suitable electronic, optical, and/or mechanical connectors to facilitate securing laser module 212 to handle 204 and providing electronic connections from laser sources 216, 218 to laser fibers extending distally from handle and/or a shaft of medical device 202. For example, laser module 212 may include an optical or electronic connector 290 configured to be removably coupled to a complementary optical or electronic connector 292 of handle 204 to operably couple laser module 212 to laser fibers within handle 204 and/or a shaft coupled to handle 204.

Optionally, laser module 212 may include laser fibers 248, 260 coupled to corresponding laser sources 216, 218 via couplers 220, 222. Laser fibers 248, 260 may be coupled or configured to be coupled to corresponding laser fibers extending through a shaft (e.g., similar to shaft 106 of medical device 102).

Imaging module 214 includes a PCB 262 and is configured to be attached to handle 204 via complementary electronic connectors, e.g., to provide electronic communication between PCB 262 and/or other components of imaging module 214 and imaging components of medical device 202. Imaging module 214 may include an electronic connector 295 configured to be removably coupled to an electronic connector 297 of handle 204 (e.g., complementary pins 293 and holes 291).

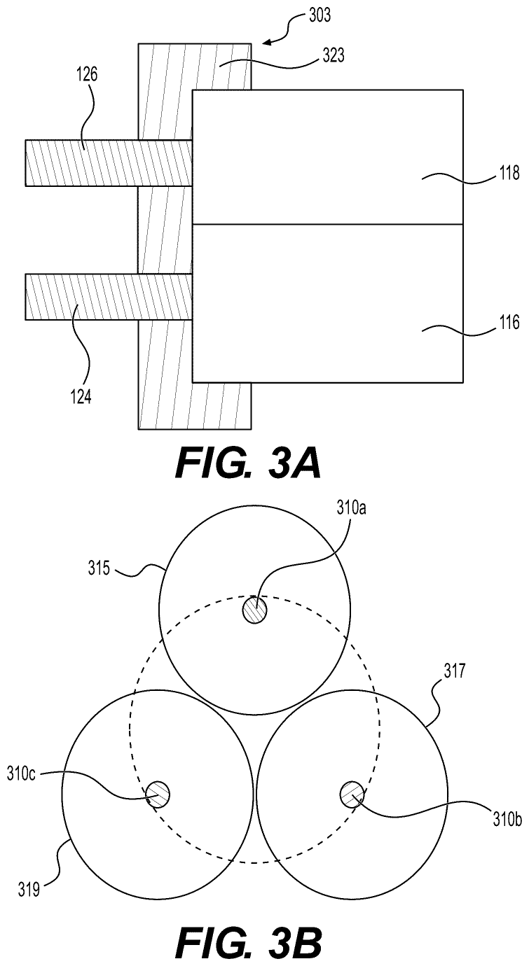

FIGS. 3A and 3B illustrate a schematic view of an exemplary component 303 that may be used to select among different wavelengths of light for the lasers in the devices herein, and that may be used in combination with any of the features of medical devices 102, 202. As mentioned above, each laser source 116, 118 of laser module 112 may be configured to emit different wavelengths, e.g., different wavelengths of visible light such as a first wavelength 315, second wavelength 317, and/or third wavelength 319. For example, first wavelength 315 may be red (e.g., 630-670 nm), second wavelength 317 may be green (e.g., 532 nm), and third wavelength 319 may be blue (e.g., 400-500 nm). Component 303 may allow an operator to select the wavelength of light emitted by each laser of the medical device.

As shown in FIG. 3A, component 303 includes an actuator, e.g., a user element 323, such as a tunable or rotatable knob. User element 323 may be incorporated into handle 104, e.g., coupled to housing 110, of medical device 102, and accessible to the hand of the operator during use of the device. User element 323 may be coupled to laser sources 116, 118 in order to adjust and/or control the wavelength(s) of light emitted. For example, an operator may actuate user element 323 to select a set of two wavelengths from a total of three wavelengths. For example, an operator may actuate user element 323 (e.g., rotate the knob) to select red and green (illustrated in FIG. 3B as settings 310a and 310b, respectively), green and blue (illustrated in FIG. 3B as settings 310b and 310c, respectively), or blue and red (illustrated in FIG. 3B as settings 310c and 310a, respectively).

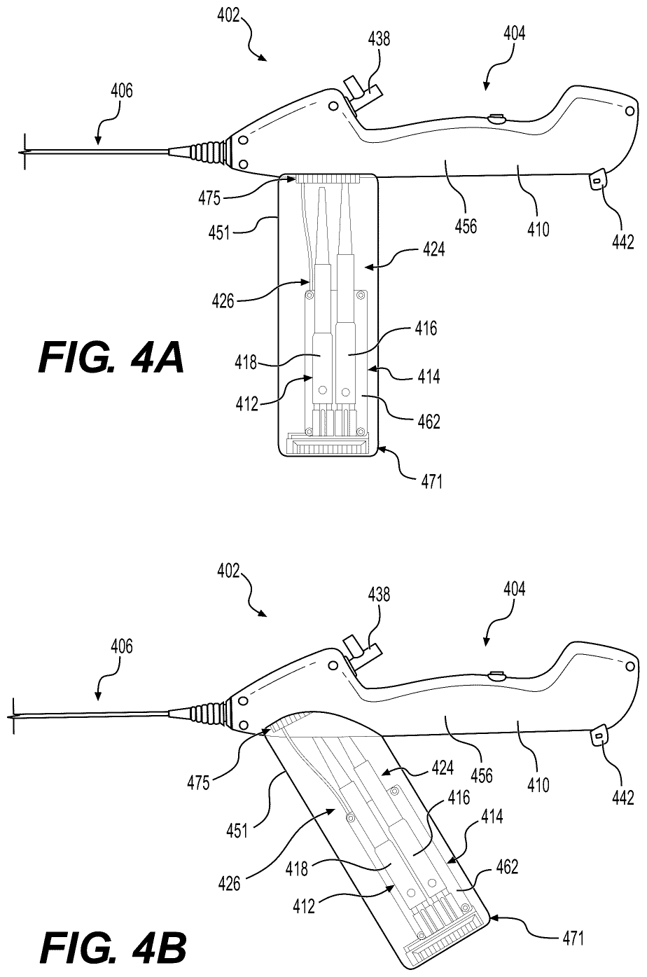

According to some examples herein, the imaging module and the laser module may be detachable from the handle via the same detachable unit. This is illustrated in FIGS. 4A and 4B. In particular, FIGS. 4A and 4B show two different versions of another exemplary medical device 402 according to aspects of the present disclosure, including a handle 404 coupled to a shaft 406. Handle 404 may include any of the features of handles 104, 204, and shaft 406 may include any of the features of shaft 106, unless otherwise specified herein. Handle 404 includes a housing 410, a port 438, an actuator 442, and a grip 456.

Medical device 402 also includes a laser module 412 and an imaging module 414 contained within a single unit 451 that is attachable to handle 404 for use of medical device 402 and detachable from handle 404 while not in use. FIG. 4A shows a configuration wherein the unit 451 is attachable to the handle 404 in a substantially perpendicular orientation, whereas FIG. 4B shows the unit 451 attachable to the handle 404 so as to form an acute angle with housing 410 of handle 404. For example, the unit 451 may form an angle of 90 degrees or less relative to a longitudinal axis of the housing 410. Other configurations and orientations are contemplated. Laser module 412 may include a first laser source 416 and a second laser source 418 coupled to a first laser fiber 424 and a second laser fiber 426, respectively. Laser fibers 424, 426 may be coupled or configured to be coupled to corresponding laser fibers extending through shaft 406 and/or housing 410. Optionally, laser module 412 may include laser sources 416, 418 configured to couple to corresponding laser fibers extending through shaft 406 and/or housing 410.

Imaging module 414 includes a PCB 462 configured to couple to imaging components (e.g., an imaging device and light source similar to imaging device 146 and light source 154 of medical device 102).

Unit 451 may be configured to selectively attach and detach from housing 410 of handle 404 so as to not interfere with operation of medical device 402. For example, as shown in FIGS. 4A and 4B, unit 451 may attach to a portion of housing 410 distal to grip 456. In other examples, unit 451 may attach to a portion of housing 410 proximal to grip 456. Although unit 451 is illustrated as having a substantially rectangular shape, it will be appreciated that unit 451 may be any other shape and/or size suitable to contain laser module 412 and imaging module 414.

Unit 451 may include an electronic connector 475 complementary to an electronic connector of handle 404 to operably couple imaging module 414 to imaging components of medical device 402 and to operably couple laser module 412 to laser components of medical device 402. Additionally or alternatively, imaging module 414 may be coupled to imaging components of medical device 402 via direct wiring. Unit 451 also may be configured to receive power from a power source and/or to couple to a display. For example, unit 451 includes an electronic connection 471 or adapter configured to receive power from an external power source and/or for electronic communication with a display or other electronic component.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

We claim:

1. A medical device comprising:
a handle comprising a housing that includes a grip and an actuator, the handle further comprising:
an imaging module comprising a printed circuit board; and
a laser module comprising a first laser source configured to couple to a first laser fiber and a second laser source configured to couple to a second laser fiber,
wherein the laser module and the imaging module are contained within the housing; or
wherein the imaging module, the laser module, or both the imaging module and the laser module are selectively attachable to and detachable from the housing.

2. The medical device of claim 1, further comprising a shaft extending from the handle to a distal tip, the distal tip comprising an imaging device and a light source each operably coupled to the imaging module.

3. The medical device of claim 2, further comprising the first laser fiber and the second laser fiber, wherein the first laser fiber is coupled to the first laser source and the second laser fiber is coupled to the second laser source, and wherein each of the first laser fiber and the second laser fiber extends distally through the shaft to the distal tip.

4. The medical device of claim 2, wherein the handle includes a port and the shaft includes a lumen in fluid communication with the port, the lumen terminating at a distalmost face of the distal tip.

5. The medical device of claim 1, wherein the laser module and the imaging module are contained within the housing, and the laser module further comprises a first coupler at least partially surrounding the first laser source and a second coupler at least partially surrounding the second laser source.

6. The medical device of claim 1, wherein the handle includes a user element selectable by a user to control a first wavelength of the first laser source and a second wavelength of the second laser source.

7. The medical device of claim 6, wherein the user element comprises a knob, switch, or button configured to select the first wavelength and the second wavelength, wherein the second wavelength is different from the first wavelength.

8. The medical device of claim 7, wherein the first wavelength is red light, green light, or blue light.

9. The medical device of claim 1, wherein both the imaging module and the laser module are coupled to, and detachable from, the housing.

10. The medical device of claim 1, wherein the imaging module and the laser module are contained within a single unit that is selectively attachable to and detachable from the housing.

11. The medical device of claim 10, wherein the single unit is coupled to, and detachable from, a portion of the housing distal to the grip.

12. The medical device of claim 11, wherein the single unit forms an angle of 90 degrees or less relative to a longitudinal axis of the housing when the single unit is attached to the housing.

13. The medical device of claim 1, wherein the imaging module and the laser module are contained within separate units, each unit being coupled to, and detachable from, the housing independent of the other unit.

14. A medical device comprising:
a handle comprising:
a housing including a grip, a port, and an actuator;
an imaging module coupled to, and selectively detachable from, the housing; and
a laser module coupled to, and selectively detachable from, the housing, the laser module comprising a first laser source and a second laser source; and
a shaft extending from the handle to a distal tip, the shaft defining a lumen in fluid communication with the port;
wherein the actuator is configured to control movement of the shaft.

15. The medical device of claim 14, wherein the distal tip of the shaft includes an imaging device and a light source each operably coupled to the imaging module, and wherein the lumen terminates at the distal tip.

16. The medical device of claim 15, wherein the first laser source is coupled to a first laser fiber and the second laser source is coupled to a second laser fiber, each of the first laser fiber and the second laser fiber extending through the shaft to respective openings of the distal tip.

17. The medical device of claim 14, wherein the imaging module and the laser module are contained within separate units, each unit being independently detachable from the housing.

18. The medical device of claim 14, wherein the imaging module and the laser module are contained within a single unit coupled to, and detachable from, the housing.

19. A medical device comprising:
a handle comprising:
a housing including a grip and an actuator;
an imaging module coupled to, and selectively detachable from, the housing; and
a laser module coupled to, and selectively detachable from, the housing, the laser module comprising a first laser source and a second laser source, and
a shaft extending from the handle to a distal tip that includes an imaging device and a light source;
wherein the imaging device and the light source are operably coupled to the imaging module; and
wherein the actuator is configured to control movement of the shaft.

20. The medical device of claim 19, wherein the first laser source is coupled to a first laser fiber and the second laser source is coupled to a second laser fiber, each of the first laser fiber and the second laser fiber extending through the shaft to the distal tip.
